(19) United States
(12) Patent Application Publication
(10) Pub. No.: US 2004/0192983 A1
(43) Pub. Date: Sep. 30, 2004

(54) Acetylene hydrogenation catalyst with segregated palladium skin

(75) Inventors: Joseph J. Bergmeister, Kingwood, TX (US); Tin-Tack P. Cheung, Kingwood, TX (US); Gary A. Delzer, Galveston, TX (US)

Correspondence Address: JENKENS & GILCHRIST, 1401 MCKINNEY, SUITE 2600, HOUSTON, TX, 77010-4034, US

(73) Assignee: Chevron Phillips Chemical Co., The Woodlands, TX

(21) Appl. No.: 10/775,913

(22) Filed: Feb. 10, 2004

Related U.S. Application Data
(60) Provisional application No. 60/448,426, filed on Feb. 18, 2003.

(51) Int. Cl. C07C 5/03; B01J 27/13

(52) U.S. Cl. 585/259; 502/224; 502/230; 502/330; 585/275

(57) ABSTRACT

A catalyst for the selective hydrogenation of acetylene comprising a support having a uniformly round external surface; palladium in the range of about 0.01 to 1.0 weight percent of the catalyst and substantially all of the palladium being concentrated in a skin periphery of the catalyst; and silver in the range of about 0.5 to 10.0 times the weight of the palladium. Preferably, the support is selected from the group consisting of alumina, titania, zirconia, zinc aluminate, zinc titanate and mixtures thereof, and/or the skin has a thickness less than about 400 microns.

### CROSS REFERENCE TO RELATED APPLICATIONS

This patent application claims priority to U.S. Provisional Application No. 60/448426, filed Feb. 18, 2003, the disclosure of which is incorporated by reference in its entirety herein.

### FIELD OF THE INVENTION

This invention relates to a catalyst for the selective hydrogenation of acetylene as well as a method for making such catalyst and to a method for the selective hydrogenation of acetylene alone or in a mixture with ethylene.

### BACKGROUND OF THE INVENTION

Ethylene is a feedstock that is used in preparing value added chemicals and polymers. One route to produce ethylene is by the pyrolysis or steam cracking of refinery gases such as ethane, propane, butane, and the like. Ethylene so produced usually contains small proportions of acetylene. In polymer grade ethylene, it is generally preferred that the acetylene content be less than about 5 ppm, most preferably less than about 1 ppm.

One of the techniques that has been used in the past for reducing the amount of acetylene in an ethylene stream involves selective hydrogenation using a catalyst comprising palladium impregnated onto an inorganic support.

One consideration is that unwanted ethylene hydrogenation may increase significantly once the acetylene concentration becomes sufficiently small (e.g., 100 ppm). Also, as the temperature of the acetylene hydrogenation reaction is increased above that which gives substantial elimination of acetylene, there is a progressive increase in the amount of ethylene that is converted to ethane. Catalyst temperature increases can thus result in runaway ethylene hydrogenation.

Also, it is common in ethylene production for the amount of carbon monoxide in the effluent from an ethane cracker to vary over a large range depending on the operating conditions, the character of the feed to the cracker, and the like. In some cases, carbon monoxide can tend to poison or otherwise degrade catalyst performance. The sulfur content of process streams can similarly vary, and can similarly tend to degrade catalyst performance. It may thus be desirable for catalyst configurations in such environments to be able to accommodate fluctuations in carbon monoxide and sulfur compounds.

Another consideration is that where a spike of carbon monoxide is present in a reactor, it may be desirable to increase the reactor temperature to compensate for a loss in catalyst activity. However, when the spike is no longer present, the higher temperature can result in runaway ethylene hydrogenation.

There is a need in the art for technologies and associated processes relating to process and economic considerations including the foregoing.

### SUMMARY OF THE INVENTION

In one aspect, the invention relates to a catalyst for the selective hydrogenation of acetylene. The catalyst comprises a support containing palladium and silver and having a uniformly round external surface; the palladium in the range of about 0.01 to 1.0 weight percent of the catalyst and substantially all of the palladium being concentrated in a skin periphery of the catalyst; and the silver in the range of about 0.5 to 10.0 times the weight of the palladium and the silver being distributed throughout the catalyst. Preferably, the support is selected from the group consisting of alumina, titania, zirconia, zinc aluminate, zinc titanate and mixtures thereof. In some embodiments, the support has a surface area in the range of about 3 to about 10 square meters per gram, and a pore volume of about 0.24 to about 0.64 cubic centimeters per gram.

Preferably, the external surface of the support is rounded to an extent such that no portion of the surface forms an angle less than about 120 degrees with any other adjacent tangent of the surface. Other tolerances as to the roundness of the external surface of the support may also be specified. In some cases, the support may be spherical, though this is not required, and it is noted that commercial catalyst supports designated as spherical may nevertheless not be truly spherical (e.g., having corners and edges remaining after processing).

In some embodiments, the catalyst may include an alkali metal (e.g., potassium) present in the range of about 0.01 to 10 weight % of the catalyst. Certain embodiments may also include a halide present in the range of about 0.1 to 10 times the molar concentration of alkali metal present in the catalyst.

In some embodiments, as an example, the dimensions of the catalyst particles can be in the range of about 2 to about 8 millimeters.

In some embodiments, the catalyst may contain silver. As an example, the catalyst may contain a weight ratio of silver to palladium that is no greater than about 10 (e.g., in the range of about 0.5 to about 8).

In some embodiments, the catalyst is prepared by impregnating alumina particles with a solution of palladium chloride or palladium nitrate. In certain embodiments, the catalyst may be prepared by mixing the catalyst particles with an aqueous solution of silver nitrate.

In some embodiments, a temperature difference between a cleanup temperature and a runaway temperature of the catalyst is greater than 50° F., and a selectivity of the catalyst for the conversion of acetylene to ethylene is greater than 40 % (e.g., greater than 50%).

In another aspect, the invention relates to a method for the treatment of a gaseous mixture comprising acetylene and optionally ethylene. For example, such a method can include:

selectively hydrogenating the acetylene therein by contacting the mixture together with hydrogen with a catalyst described herein;

In some embodiments, the gaseous mixture contains less than about 1000 ppm of carbon monoxide (e.g., less than about 600 or 400 ppm of carbon monoxide).

In some embodiments, the hydrogenation temperature can be in the range of about 35° C. to about 150° C. and the space velocity can be in the range of about 1,000 to 20,000 hr⁻¹.

Certain embodiments may include processes according to such methods, wherein the catalyst is housed in a vessel, and wherein such processes comprise steps including:

flowing the acetylene through the vessel to contact the catalyst;

flowing a heat transfer fluid (e.g., a closed loop fluid or another process stream at a desired temperature) across an exterior surface of the vessel to remove heat from the vessel; and

modulating the flow of heat transfer fluid to maintain a temperature of the heat transfer fluid within a predetermined range.

Various embodiments of such methods and processes may further include any of the other aspects and features described herein.

Additional aspects, features and advantages of embodiments under the invention will become apparent from review of the detailed description and the claims.

### DESCRIPTION OF EMBODIMENTS OF THE INVENTION

In the following description, all numbers disclosed herein are approximate values, regardless whether the word “about” or “approximate” is used in connection therewith. They may vary by 1 percent, 2 percent, 5 percent, or, sometimes, 10 to 20 percent. Whenever a numerical range with a lower limit, R_{L} and an upper limit, R_{U}, is disclosed, any number falling within the range is specifically disclosed. In particular, the following numbers within the range are specifically disclosed: R=R_{L}+k*(R_{U}—R_{L}), wherein k is a variable ranging from 1 percent to 100 percent with a 1 percent increment, i.e., k is 1 percent, 2 percent, 3 percent, 4 percent, 5 percent, . . . , 50 percent, 51 percent, 52 percent, . . . , 95 percent, 96 percent, 97 percent, 98 percent, 99 percent, or 100 percent. Moreover, any numerical range defined by two R numbers as defined in the above is also specifically disclosed.

Embodiments of the invention provide a catalyst and a method for the selective hydrogenation of acetylene. The catalyst comprises a support containing palladium and silver and having a uniformly round external surface; the palladium in the range of about 0.01 to 1.0 weight percent of the catalyst and substantially all of the palladium being concentrated in a skin periphery of the catalyst; and the silver in the range of about 0.5 to 10.0 times the weight of the palladium. Preferably, the support is selected from the group consisting of alumina, titania, zirconia, zinc aluminate, zinc titanate and mixtures thereof. In some embodiments, the skin has a thickness less than about 400 microns. The term “substantially all of the palladium” used herein means at least 90 percent of the total palladium. Preferably, at least 95 percent of the total palladium is in the skin periphery of the catalyst. In some embodiments, at least 99 percent of the total palladium is in the skin periphery of the catalyst

In the context of the present invention, the term “catalyst” refers to the support together with all materials contained or impregnated in or on the support. The palladium can be about 0.01 to about 1.0 weight percent of the catalyst. The weight percent silver can be at least half that of the palladium. In some cases, the catalyst can be further characterized in that at least 90 weight percent of the catalyst particles have the palladium concentrated in an area within 400 microns of the exterior surface while the silver is distributed throughout the particles. In the context of this invention, the restriction of the palladium to the skin is referred to as a segregated palladium skin (i.e., the palladium is segregated from the rest of the catalyst particle). The amount of silver in the catalyst is generally not more than about 10 times that of the palladium, e.g., in the range of about 0.5 to about 8 times that of the palladium. For further reference, the teachings of U.S. Pat. Nos. 4,404,124 and 4,484,015 are hereby incorporated by reference.

The term “skin” used herein refers to the exterior surface of the catalyst composition which contain components, such as palladium, of the catalyst composition. The skin can be any thickness as long as such thickness can promote the hydrogenation process disclosed herein. Generally, the thickness of the skin can be in the range of from about 1 micron to about 1000 microns, or from about 5 microns to about 750 microns, or from about 5 microns to about 500 microns, or from 10 microns to 300 microns. In some embodiments, the skin thickness is less than 1000 microns, less than 750 microns, less than 500 microns, less than 400 microns, or less than 300 microns.

One can use any suitable method to determine the concentration of the palladium in the skin of the catalyst composition. Determining the concentration of the palladium in the skin of the catalyst composition also helps in determining the thickness of the skin. One technique currently employed is the electron microprobe which is known to one skilled in the art. Another technique involves breaking open a representative sample of the catalyst composition (in catalyst particle form) and treating the catalyst particles with a dilute alcoholic solution of N,N-dimethyl-para-nitrosoaniline. The treating solution reacts with the palladium to give a red color which can be used to evaluate the distribution of the palladium. Another technique for measuring the concentration of the palladium in the skin of the catalyst composition involves breaking open a representative sample of catalyst particles followed by treatment with a reducing agent such as hydrogen, to change the color of the skin to evaluate the distribution of the palladium.

The support is selected to provide surface area in the range of about 3 to about 10 square meters per gram, pore volume of about 0.24 to about 0.64 cubic centimeters per gram. In some embodiments, the surface area of the support is less than 6 square meters per gram, less than 5 square meters per gram, less than 4 square meters per gram, or less than 3 square meters per gram. In other embodiments, the pore volume is less than 0.54 cubic centimeters per gram, less than 0.44 cubic centimeters per gram, or less than 0.34 cubic centimeters per gram. It may also range from about 0.24 to about 0.34 cubic centimeters per gram or from about 0.35 to about 0.64 cubic centimeters per gram. Depending on the desired results, the surface area and pore volume of a catalyst support can be higher or lower than the values given above.

In addition, the average pore diameter of the support generally ranges from a few hundred Angstroms to several thousand Angstroms. In some embodiments, the average pore diameter is greater than 600 Angstroms, greater than 700 Angstroms, greater than 800 Angstroms, or greater than 900 Angstroms. In other embodiments, the average pore diameter is less than 5,000 Angstroms, less than 4000 Angstroms, or less than 3000 Angstroms. In some other embodiments, the average pore diameter may range from about 700 Angstroms to about 5000 Angstroms, from about 900 Angstroms to about 3000 Angstroms, or from about 1200 Angstroms to about 2500 Angstroms.

These characteristics of the support can be determined using the following methods on samples of the support that has been degassed at room temperature for 30 minutes at a pressure of 10⁻³ mm or less.

The surface area can be measured by the well-known method of Brunauer, Emmett, and Teller (“BET”) by measuring the quantity of argon adsorbed on the catalyst at −183° C. with the cross-sectional area of the argon atom being taken as 14.4 square Angstrom units. Alternatively, it can also be measured by mercury intrusion. One such a method is described in ASTM UOP 578-02, entitled “Automated Pore Volume and Pore Size Distribution of Porous Substances by MERCURY Porosimetry,” which is incorporated by reference herein in its entirety.

Determining the pore volume involves determining the “mercury density” and the “helium density”. The mercury density is determined by immersing the support in mercury at 20° C. and 900 mm pressure, under which conditions about 15 minutes are allowed for attainment of equilibrium. The helium density is determined by immersing the support in helium at room temperature. The pore volume per gram is found by subtracting the reciprocal of the “helium density” from the reciprocal of the “mercury density.”

The mean pore radius can be roughly estimated by the formula r=(2V/A), where r is the mean pore radius, V is the pore volume, and A is the surface area. If V is expressed in cubic centimeters and A is expressed in square centimeters, the mean radius r is in centimeters and should be multiplied by 10⁸ to give the mean radius in Angstrom units. The above equation assumes cylindrical pores. When the catalyst includes non-cylindrical pores and/or cracks, the mean pore radius may deviate from the one provided by the above equation.

The exterior surface of the support is uniformly round, meaning that its surface is generally round, having no corners. A primary consideration is that the palladium skin thickness may tend to be thicker at any such corners and edges, such that the selectivity of the catalyst is degraded. The uniform round surface of the support generally results in a more uniform skin thickness. Another consideration is that some spherical supports available in the industry may have been prepared from rolling tablets or cubes or by other processes, and may have residual edges (even though designated as spherical). Therefore, the uniform round surface of the support is substantially free of corners or edges. In some embodiments, a uniformly round catalyst support encompasses spherical particles; however, in other embodiments, a uniformly round catalyst support include only those non-spherical structures which are uniformly round. Such structures includes, but are not limited to, ovals, egg-shaped objects, and soccer-ball-shaped objects, etc. While uniformly round structures do not include a cylinder, it does include a cylindrical structure with its edges rounded off.

The uniform roundness of the support may be further defined in this context in terms of contact angles along the exterior of the support. For example, it may be desired that no portion of the exterior surface of the support (e.g., an apex of a protrusion) form an angle of less than 140 degrees with any other adjacent tangent of the exterior support. In this context, the portion and adjacent tangent forming the angle of measure are adjacent, meaning they have no space between them, such that the apex of the angle formed between the portions is located on the surface of the support. Other tolerances on the roundness of the support can also be specified, such as 130 degrees or 120 degrees in the above measure. Similarly, it may be desirable that the skin thickness of the catalyst be within a certain tolerance (e.g., less than about 400 microns). Subject to such constraints, the shape of the support under the present invention does not have to be spherical, it merely needs to have a near uniformly round surface with no corners as defined above. In other embodiments, a spherical or nearly spherical support is used.

As mentioned above, the uniform roundness of the support may also be characterized as having a substantially uniform skin thickness. This means that the skin thickness should not vary by more than 70% throughout the external surface of the catalyst. In some embodiments, the variation in skin thickness throughout the catalyst should be less than 50%, or less than 40%, or less than 30%. The term “variation” refers to the deviation in thickness from the average skin thickness.

The palladium can be placed on the alumina in any suitable manner that will yield a catalyst meeting the above-described parameters. As an example, the alumina can be impregnated with an aqueous solution of palladium chloride.

It may be desired that a collection of catalyst particles have a degree of uniformity with regard to the characteristics discussed above. For example, it may be desired that at least 90 weight percent of the catalyst particles have the palladium concentrated in an area within 400 microns of the exterior surface. One technique for such a determination involves breaking open a representative sample of catalyst particles and treating them with a dilute alcoholic solution of N,N-dimethyl-para-nitrosoaniline. The treating solution reacts with the palladium to give a red color which can be used to evaluate the distribution of the palladium.

The size of the catalyst particles can be tailored for a given application, and a tolerance for the size distribution of a collection of catalyst particles may be similarly tailored. As an example, it may be desirable to employ catalyst particles having minimum dimensions of at least about 1 millimeter, e.g., having dimensions in the range of about 2 to about 8 millimeters.

In some embodiments, the silver can be distributed throughout the catalyst in any suitable manner. As an example, the catalyst particles can be placed in an aqueous silver nitrate solution of a quantity greater than that necessary to fill the pore volume of the catalyst particles. The impregnated catalyst is dried at a temperature in the range of about 25° C. to about 150° C. In other embodiments, the silver is not substantially present in the palladium skin. For example, no more than 80 weight percent of the silver is present in the palladium skin. In some instances, the silver is present in the palladium skin periphery by less than 70 weight percent, less than 60 weight percent, or less than 50 weight percent. In some other instances, the silver is present in the palladium skin periphery by less than 40 weight percent, less than 30 weight percent, or less than 20 weight percent. In still other instances, the silver is present in the palladium skin by less than 10 weight percent, such as about 1%, 3%, 5%, 7% or 9%. Some catalysts is substantially free of silver in the palladium skin periphery.

The dried catalyst can be employed directly as a catalyst for hydrogenation. It may also be calcined to decompose the compounds providing the palladium and silver. As an example, this calcining can be done at temperatures up to about 600° C. (e.g., in the range of 150° C. to 550° C.). The calcining may also be followed by a reduction step. This reduction can be accomplished using the feed for the selective hydrogenation. The catalyst may also be reduced with a gas such as hydrogen since optimum operation of the selective hydrogenation does not begin until there has been reduction of the catalytic metals. As an example, the reduction can be carried out at a temperature in the range of about 25° C. to about 450° C.

In some embodiments, the catalyst may further include an alkali metal component and a halide component to tailor the activity of the catalyst for a given application. For example, the supported Pd/Ag catalyst material can be impregnated with an aqueous solution of at least one alkali metal hydroxide and/or at least one alkali metal fluoride (e.g., KOH and/or KF), followed by drying (generally at about 50° C.-150° C.) and calcining (e.g., in air at a temperature of about 400° C.-600° C.) for about 1-6 hours. It may be desired to have the alkali metal component of the catalyst present in a range of about 0.01 to 10 weight % of the catalyst. It may also be desired to have the ratio of the halide to alkali metal component of the catalyst present in a ratio of about 1:0.11 to 1:10. For further reference, the teachings of U.S. Pat. Nos. 5,583,274 and 5,587,348 are hereby incorporated by reference.

It may also be desirable to further employ a “wet-reducing” agent present during the contacting of the supported Pd/Ag catalyst with the alkali metal hydroxide and/or the alkali metal fluoride. Non-limiting examples of such “wet-reducing” agents are: hydrazine, at least one alkali metal borohydride, at least one aldehyde containing 1-6 carbon atoms per molecule such as formaldehyde, at least one ketone containing 1-6 carbon atoms per molecule, at least one carboxylic acid containing 1-6 carbon atoms per molecule such as formic acid or ascorbic acid, at least one reducing sugar containing an aldehyde or alpha-hydroxyketone group such as dextrose, and the like.

The selective hydrogenation is carried out by passing the gas stream of ethylene, containing the acetylene to be removed, along with hydrogen into contact with the catalysts. In order to best approach substantially complete removal of the acetylene, there should be at least one mole of hydrogen for each mole of acetylene.

The temperature necessary for the selectivity hydrogenation may depend upon the activity of the catalyst and the extent of acetylene removal desired. As an example, temperatures in the range of about 35° C. to about 100° C. may be used. Any suitable reaction pressure can be employed. Generally, the total pressure is in the range of about 100 to about 1,000 pounds per square inch gauge. The gas hourly space velocity (GHSV) can also vary over a wide range. Typically, the space velocity will be in the range of about 1,000 to about 15,000 liters of feed per liter of catalyst per hour.

Regeneration of the catalyst may be accomplished by heating the catalyst in air at a temperature preferably not in excess of 500° C. to burn off any organic matter, polymer, or char.

A further understanding of the present invention and its advantages will be provided by the following examples. The following examples are presented to exemplify embodiments of the invention. All numerical values are approximate. When numerical ranges are given, it should be understood that embodiments outside the stated ranges may still fall within the scope of the invention. Specific details described in each example should not be construed as necessary features of the invention.

### EXAMPLE I

This example illustrates the preparation of various palladium-containing catalyst compositions to be used in a hydrogenation process.

Catalyst A (Control) was a commercial Pd/Ag/Al₂O₃ catalyst in the form of 4 mm×4 mm tablets which contained 0.016 weight %Pd and 0.041 weight % Ag and about 99 weight % Al₂O₃. It had a surface area of about 3-5m²/g (determined by BET method employing N₂) and had been provided by Sud Chemie of Louisville, Ky., USA under the product designation G-83C.

Catalyst B (Control) contained 0.018 weight % Pd and 0.062 weight % Ag and about 99 weight % Al₂O₃. The catalyst was prepared as described in USP 4,404,124 on 4 mm×4 mm tablets of alpha-aluminum oxide having a surface area of 3-7m²/g (determined by BET method employing N₂)

Catalyst C (Invention) contained 0.018 weight % Pd and 0.052 weight % Ag and about 99 weight % Al₂O₃. The catalyst was prepared as described in U.S. Pat. No. 4,404,124 on 5×8 mesh spheres of alpha-aluminum oxide having a surface area of 3-7m²/g (determined by BET method employing N₂)

Catalyst D (control) contained 0.018 weight % Pd, 0.062 weight % Ag, and 0.3 weight % K and about 99 weight % Al₂O₃. The catalyst was prepared by adding potassium hydroxide via incipient wetness to catalyst B, followed by calcination at 538° C. for 3 hours in air. For further reference, the teachings of U.S. Pat. Nos. 5,489,565, 5,488,024, and 5,510,550 are hereby incorporated by reference.

Catalyst E (Invention) contained 0.018 weight % Pd, 0.052 weight % Ag, and 0.3 weight % K and about 99 weight % Al₂O₃. The catalyst was prepared by adding potassium hydroxide via incipient wetness to catalyst C.

### EXAMPLE II

This example illustrates the performance of the catalysts described hereinabove in Example I in a hydrogenation process.

Hydrogenation runs were made with 20 cc of each of the above described catalysts. The catalysts were placed in a stainless steel reactor tube having a 0.62 inch inner diameter and a length of about 18 inches. The catalyst (resided in the middle of the reactor; both ends of the reactor were packed with 6 mL of alundum) was reduced at about 100° F. for about 1 hour under hydrogen gas flowing at 200 mL/min at 200 pounds per square inch gauge (psig). Thereafter, a hydrocarbon-containing fluid, typical of a feed from the top of a de-ethanizer fractionation tower in an ethylene plant, containing approximately (all by weight unless otherwise noted) hydrogen, 2.1%; methane, 22%; ethylene, 70%; acetylene, 3500 ppm; carbon monoxide, 300 ppm was introduced into the reactor at a flow rate of 900 mL per minute at 200 psig translating to a gas hourly space velocity of about 2700 hour⁻¹. The reactor temperature was increased until the hydrogenation ran away, i.e., the uncontrollable hydrogenation of ethylene was allowed to occur. The reactor was then allowed to cool to room temperature before data collection was started.

Feed (900 mL/min @200 psig) was passed over the catalyst while holding the temperature constant before sampling the exit stream by gas chromatography. The catalyst temperature was determined by inserting a thermocouple into a thermowell running the length of the reactor and varying its position until the highest temperature was observed, the furnace was then raised a few degrees, and the testing cycle was repeated until 3 weight % of ethane was produced.

The cleanup temperature, T1, is defined as the temperature at which the acetylene concentration drops below 20 ppm. The T2, runaway temperature, is defined as the temperature at which 3 wt % of ethane is produced. At this temperature the uncontrolled hydrogenation of ethylene to ethane begins. And delta T is the difference between T2 and T1. This value can be viewed as a measure of selectivity or even a window of operability.

Table 1 contains data from runs using a commercial and laboratory prepared control as well as the invention catalyst. All catalyst had a silver to palladium ratio of 3.

TABLE 1

| | | | catalyst | Pd in Reactor, | T1 | ΔT | Selectivity |
|---|---|---|---|---|---|---|---|
| Run # | catalyst | shape | charge, g | mg | ° F. | ° F. | to C₂H₄, % |
| 201(control) | A | pellet | 21.94 | 3.53 | 96 | 44 | 27.0 |
| 202(control) | B | pellet | 20.9 | 3.85 | 105 | 46 | 37.9 |
| 203(invention) | C | sphere | 14.0 | 2.51 | 105 | 53 | 49.0 |

Table 2 below shows the surface area, pore diameter, and pore volume for each of the above catalysts.

TABLE 2

| | Surface | Average Pore | Pore Diameter | Pore |
|---|---|---|---|---|
| Sample | Area | Diameter | Range | Volume |
| Number | (m²/g) | (nm) | (nm) | (mL/g) |
| 201 | 3.75 | 198 | 60-1250 | 0.257 |
| 202 | 4.86 | 167 | 70-1200 | 0.276 |
| 203 | 3.86 | 271 | 110-1100 | 0.517 |

As can be seen in table 1 the total catalyst charge of the spherical catalyst was less than the catalysts made on pelletized support. Comparing run 201 to 202 one can observe that the operating window or delta T are approximately the same, however the delta T made on the sphere catalyst has a delta T significantly higher than the two controls. Also the selectivity to ethylene is greater than both controls.

### Example III

Hydrogenation runs were performed as described in Example 2.

TABLE 3

| | | | catalyst | Pd in | T1 | ΔT | Selectivity |
|---|---|---|---|---|---|---|---|
| Run # | catalyst | shape | charge, g | Reactor, mg | ° F. | ° F. | to C₂H₄, % |
| 301(control) | D | pellet | 21.63 | 3.98 | 105 | 53 | 47 |
| 302(invention) | E | sphere | 14.51 | 2.61 | 103 | 51 | 56 |

As can be seen in table 3 the total catalyst charge of the spherical catalyst was less than the catalysts made on pelletized support. Comparing run 301 to 302 one can observe that the operating window or delta T are approximately the same, however the selectivity to ethylene on the sphere catalyst is significantly higher than the control. Also the total amount of palladium in the reactor is significantly smaller for the sphere catalyst than the control which would give the sphere catalyst a large economic benefit.

While the invention has been described with respect to a limited number of embodiments, the specific features of one embodiment should not be attributed to other embodiments of the invention. No single embodiment is representative of all aspects of the inventions. In some embodiments, the compositions may include numerous compounds not mentioned herein. In other embodiments, the compositions do not include, or are substantially free of, any compounds not enumerated herein. Variations and modifications from the described embodiments exist. The method of making the catalysts is described as comprising a number of acts or steps. These steps or acts may be practiced in any sequence or order unless otherwise indicated. Finally, any number disclosed herein should be construed to mean approximate, regardless of whether the word “about” or “approximately” is used in describing the number. The appended claims intend to cover all those modifications and variations as falling within the scope of the invention.

What is claimed is:

1. A catalyst for the selective hydrogenation of acetylene, comprising:
a support selected from the group consisting of alumina, titania, zirconia, zinc aluminate, zinc titanate and mixtures thereof, wherein the support has a uniformly round external surface, a surface area in the range of about 3 to about 10 square meters per gram, and a pore volume of about 0.24 to about 0.64 cubic centimeters per gram;
palladium in the range of about 0.01 to 1.0 weight percent of the catalyst, wherein substantially all of the palladium is concentrated in a skin periphery of the catalyst, wherein the skin has a thickness less than about 400 microns; and
silver in the range of about 0.5 to 10.0 times the weight of the palladium, wherein the silver is distributed throughout the catalyst.

2. A catalyst according to claim 1, wherein the external surface of the support is rounded to an extent such that the skin thickness is substantially uniform throughout the external surface.

3. A catalyst according to claim 1, wherein the support of the catalyst is non-spherical yet uniformly round.

4. A catalyst according to claim 3, wherein non-spherical support is oval-shaped, egg-shaped, or soccer-ball-shaped.

5. A catalyst according to claim 1, wherein the support of the catalyst is spherical and has an average pore diameter from about 600 Angstroms to about 5000 Angstroms.

6. A catalyst according to claim 1, further comprising:
an alkali metal present in the range of about 0.01 to 10 weight % of the catalyst.

7. A catalyst according to claim 6, further comprising:
a halide in the range of about 0.1 to 10 times the molar concentration of alkali metal present in the catalyst.

8. A catalyst according to claim 7, wherein the alkali metal comprises potassium.

9. A catalyst according to claim 1, wherein the dimensions of the catalyst particles are in the range of about 2 to about 8 millimeters.

10. A catalyst according to claim 9, wherein the weight ratio of silver to palladium is no greater than about 10.

11. A catalyst according to claim 9, wherein the weight ratio of silver to palladium is in the range of about 0.5 to about 8.

12. A catalyst according to claim 11, containing about 0.01 to 0.5 weight percent palladium.

13. A catalyst according to claim 1, wherein the catalyst is prepared by impregnating alumina particles with a solution of palladium chloride or palladium nitrate.

14. A catalyst according to claim 13, wherein the catalyst is prepared by mixing the catalyst particles with an aqueous solution of silver nitrate.

15. A catalyst according to claim 1, wherein a selectivity of the catalyst for the conversion of acetylene to ethylene is greater than 40%.

16. A catalyst according to claim 15, wherein the palladium is less than 0.05 weight % of the catalyst.

17. A catalyst according to claim 7, wherein a selectivity of the catalyst for the conversion of acetylene to ethylene is greater than 50%.

18. A catalyst according to claim 17, wherein the palladium is less than 0.03 weight % of the catalyst.

19. A method for the treatment of a gaseous mixture comprising acetylene, which method comprises selectively hydrogenating the acetylene therein by contacting the mixture together with hydrogen with a catalyst;
wherein the catalyst comprises a support selected from the group consisting of alumina, titania, zirconia, zinc aluminate, zinc titanate, and mixtures thereof, wherein the support has a uniformly round external surface, a surface area in the range of about 3 to about 10 square meters per gram, and a pore volume of about 0.24 to about 0.64 cubic centimeters per gram;
wherein the catalyst comprises palladium in the range of about 0.01 to 1.0 weight percent of the catalyst, wherein substantially all of the palladium is concentrated in a skin periphery of the catalyst, wherein the skin has a thickness less than about 400 microns; and
wherein the catalyst comprises silver in the range of about 0.5 to 10.0 times the weight of the palladium, wherein the silver is distributed throughout the catalyst.

20. A method according to claim 19, wherein the external surface of the support is rounded to an extent such that the skin thickness is substantially uniform throughout the external surface.

21. A method according to claim 19, wherein the support of the catalyst is non-spherical yet uniformly round.

22. A method according to claim 21, wherein the non-spherical support is oval-shaped, egg-shaped, or soccer-ball-shaped.

23. A method according to claim 19, wherein the external surface of the support is spherical and has an average pore diameter from about 600 Angstroms to about 5000 Angstroms.

24. A method according to claim 19, wherein the catalyst further comprises an alkali metal present in the range of about 0.01 to 10 weight % of the catalyst.

25. A method according to claim 24, wherein the catalyst further comprises a halide in the range of about 0.1 to 10 times the molar concentration of alkali metal present in the catalyst.

26. A method according to claim 25, wherein the alkali metal comprises potassium.

27. A method according to claim 19, wherein the gaseous mixture contains less than about 1000 ppm of carbon monoxide.

28. A process according to claim 27, wherein the weight ratio of silver to palladium in the catalyst is no greater than about 10.

29. A method according to claim 28, wherein the dimensions of the catalyst particles are in the range of about 2 to about 8 milliliters.

30. A method according to claim 29, wherein the weight ratio of silver to palladium is in the range of about 0.5 to about 8.

31. A method according to claim 30, wherein the hydrogenation temperature is in the range of about 35° C. to about 150° C. and the space velocity is in the range of about 1,000 hr⁻¹ to about 20,000 hr⁻¹.

32. A method according to claim 31, wherein the gaseous mixture contains no more than about 800 ppm of carbon monoxide.

33. A method according to claim 32, wherein the catalyst is prepared by impregnating alumina particles with a solution of palladium chloride, calcining the impregnated alumina particles, and mixing the particles with an amount of an aqueous solution of silver nitrate in excess of the pore volume of the alumina.

34. A process according to claim 33, wherein the catalyst contains about 0.01 to 10 weight % palladium.

35. A process according to claim 19, wherein the catalyst is housed in a vessel, further comprising:
flowing the acetylene through the vessel to contact the catalyst;
flowing a heat transfer fluid across an exterior surface of the vessel to remove heat from the vessel; and
modulating the flow of heat transfer fluid to maintain a temperature of the heat transfer fluid within a predetermined range.

36. A process according to claim 35, wherein the predetermined range is about 30° C. to about 150° C.

37. A method according to claim 19, wherein a selectivity of the catalyst for the conversion of acetylene to ethylene is greater than 40%.

38. A method of claim 37, wherein the palladium is less than 0.05 weight % of the catalyst.

39. A method according to claim 25, wherein a selectivity of the catalyst for the conversion of acetylene to ethylene is greater than 50%.

40. A method of claim 39, wherein the palladium is less than 0.03 weight % of the catalyst.

41. A catalyst for the selective hydrogenation of acetylene, comprising:
an alpha alumina support, wherein the support has a uniformly round external surface, a surface area in the range of about 3 to about 10 square meters per gram, a pore diameter of about 600 Angstroms to about 5000 Angstroms, and a pore volume of about 0.24 to about 0.64 cubic centimeters per gram;
palladium in the range of about 0.01 to 1.0 weight percent of the catalyst, wherein substantially all of the palladium is concentrated in a skin periphery of the catalyst, wherein the skin has a thickness less than about 400 microns;
silver in the range of about 0.5 to 10.0 times the weight of the palladium, wherein the silver is distributed throughout the catalyst;
potassium present in the range of about 0.01 to 10 weight % of the catalyst; and
fluoride in the range of about 0.1 to 10 times the molar concentration of potassium present in the catalyst.

42. A catalyst according to claim 41, wherein a selectivity of the catalyst for the conversion of acetylene to ethylene is greater than 50%.

43. A catalyst according to claim 42, wherein the palladium is less than 0.05 weight % of the catalyst.
